(19) United States
(12) Patent Application Publication
(10) Pub. No.: US 2021/0033602 A1
(43) Pub. Date: Feb. 4, 2021

(54) Simplifying Solid-Phase Microextraction (SPME)-Based Analytical Measurements of Exceedingly Small-Volume Samples by Application of Negligible Depletion

(71) Applicants: Marc David Porter, Park City, UT (US); Jennifer Harnisch Granger, Salt Lake City, UT (US); Robert Joseph Soto, Thousand Oak, CA (US); Lorriane Marie Siperko, Salt Lake City, UT (US)

(72) Inventors: Marc David Porter, Park City, UT (US); Jennifer Harnisch Granger, Salt Lake City, UT (US); Robert Joseph Soto, Thousand Oak, CA (US); Lorriane Marie Siperko, Salt Lake City, UT (US)

(73) Assignee: University of Utah, Salt Lake City, UT (US)

(21) Appl. No.: 16/940,824

(22) Filed: Jul. 28, 2020

Related U.S. Application Data
(60) Provisional application No. 62/879,819, filed on Jul. 29, 2019.

(51) Int. Cl. G01N 33/543

(52) U.S. Cl. CPC G01N 33/54306

(57) ABSTRACT

This invention discloses an approach regarding the use of solid-phase microextractions (SPMEs) in the analytical, bioanalytical, combinatorial sciences, and all other applicable areas of measurement science. The approach applies to the analysis of exceedingly small volumes of a liquid specimen (10s-100s of μL), and how the concepts of negligible depletion (ND) can be used within the context of tradeoff between extractive (reaction) kinetics, extractive capacity, and sample flow rate as a means to obviate the need to deliver accurately a small volume sample for SPME analysis, improving the ease-of-use for a number of different SPME-based measurements including, for example, disease markers in immunoassays for health care.

### REFERENCE TO RELATED APPLICATION

This application claims inventions disclosed in Provisional Patent Application No. 62/879,819, filed Jul. 29, 2019, entitled “NEGLIGIBLE DEPLETION AS A MEANS TO SIMPLIFY SOLID PHASE EXTRACTION (SPE).” The benefit under 35 USC § 119(e) of the above mentioned United States Provisional Applications is hereby claimed, and the aforementioned application is hereby incorporated herein by reference.

### FIELD OF THE INVENTION

This invention generally relates to the use of solid-phase microextraction (SPME) in the analytical, bioanalytical, combinatorial, and other measurement sciences, and more specifically relates to applying the concept of negligible depletion (ND) to SPME as a means to obviate the need to deliver an accurate volume of sample.

### BACKGROUND

Solid-phase microextraction (SPME) is a sampling technique that relies on the extraction of an analyte(s) present in a liquid sample. Two embodiments of SPME include fibers modified with solid or liquid bonded phases for volatiles analysis using headspace gas chromatographic and/or mass spectrometric analysis and thin, porous, membranes, oftentimes called SPME disks or SPME membranes. Other SPME methodologies, like colorimetric solid-phase microextraction (C-SPE), enable the detection of the extracted analyte directly on the disk by, for instance, reacting the analyte with an indicator dye previously impregnated within the disk. The resulting colored product can be quantitated by diffuse reflection spectroscopy. An important aspect of SPME is that the process of extraction inherently concentrates the analyte with the potential for separation from undesirable matrix components, thereby both simplifying the measurement and increasing quantitative capability.

The recent importance of SPME is driven, at least in part, by the growing demand for rapid, low-cost, and easy-to-use analytical measurement methods that can be performed outside of a formal research laboratory setting. The realization of such capabilities will enable users to carry out measurements central to on-site environmental testing, homeland security, law enforcement, extraterrestrial exploration, point-of-care (POC) health care diagnostics, and many other technological areas. One of the operational challenges in the application of SPME to many of these technological areas is the need precisely and accurately meter specific volumes of the liquid sample through the membrane. As an example, for rapid diagnostic testing, sample volumes ranging from approximately 10 to 500 μL are desirable. However, variability in specimen composition (e.g., hematocrit and total protein content for blood specimens), the much slower uptake rates of many types of biological analytes by an SPME membrane, and possible absence sophisticated volumetric equipment can make sample delivery a significant obstacle to quantitative testing outside of a laboratory. This invention approaches this challenge by applying the principles of negligible depletion (ND) and reaction rate and equilibrium considerations as a means to obviate the need to exactingly measure and deliver a known amount of a small volume of a liquid through the membrane disk in SPME technologies.

### SUMMARY OF THE INVENTION

The goal of the present invention is to obviate the need to meter an accurate amount of a small volume (500 μL or less) of the sample through a solid-phase microextraction (SPME) membrane for the purpose of measuring one or more analytes. In so doing, the invention applies the principles of negligible depletion (ND) to the process, using, by way of an example, a sandwich immunoassay for human immunoglobulin-G protein (h-IgG) carried out on an SPME membrane, modified with anti-h-IgG capture antibody. In this context, ND requires passing sufficient samples through the SPME membrane at flow rates, which are slow enough that the binding reaction between h-IgG and corresponding capture antibody has enough time to reach equilibrium. Under these conditions, the total amount of extracted h-IgG is proportional to the concentration in the sample and becomes independent of sample volume, thereby improving ease-of-use. ND may be more generally applied to any SPME-based measurement including, for example, disease markers in immunoassays for health care.

### BRIEF DESCRIPTION OF THE FIGURES

The accompanying figures, when coupled together with the detailed descriptions presented below, serve to illustrate further various embodiments of the invention and to explain various principles and advantages associated with the present invention.

FIG. 1 is an illustrative example of a flow-through assay (FTA) cartridge-based on solid-phase microextraction (SPME) and consisting of an assay membrane, wicking pad, and protective housing;

FIG. 2 is an illustrative example of the architecture and workflow for a FTA using an SPME membrane and gold nanoparticles as the label for readout by surface-enhanced Raman spectroscopy (SERS). An antibody designed to bind the target analyte is first immobilized on a polymeric SPME membrane, followed by the application of a small volume of a test liquid specimen to the flow-through capture membrane. The sample is pulled through the membrane by the capillary action of the wicking pad, and the analyte in the sample is then selectively captured and concentrated in the membrane. The next step applies a small volume of a suspension of antibody-immobilized gold nanoparticles (AuNPs), which selectively tags the capture antigen. The response of the test is then read out with a Raman spectrometer;

FIG. 3 is an illustrative example of the results from a numerical simulation of conditions required to achieve negligible depletion in an immunoassay (antigen-binding step). Inset A of FIG. 3 plots the fractional binding for a fixed value of Γ_{Cap,0} (54.7 fmol/cm²) and C_{Ag}ⁱ (3.33 nM) for different values of Kₐ: 10⁷-10⁹ L/mol. Inset B of FIG. 3 plots the fractional binding for a fixed value of Γ_{Cap,0} (54.7 fmol/cm²) and C_{Ag}ⁱ (3.33 nM) for another set of different values for Kₐ: 10⁹-10¹¹ L/mol. Inset C of FIG. 3 are plots of V_{Ag}⁹⁵ as a function of antigen concentration (C_{Ag}ⁱ) and Kₐ at a fixed Γ_{Cap, 0}=54.7 fmol/cm²). Inset D of FIG. 3 plots V_{Ag}⁹⁵ versus Γ_{Cap,0} for fixed Kₐ of (10⁹ L/mol) and C_{Ag}ⁱ (1.67 nM); and

FIG. 4 is an illustrative example of the response for an SPME membrane-based immunoassay for h-IgG with SERS readout. Goat anti-human IgG is immobilized onto a nitrocellulose SPME disk and exposed to different volumes of 100 ng/mL human IgG. A subsequent labeling step with antibody-conjugated gold nanoparticles (goat anti-human IgG) is performed under the same conditions for all samples. Prior to the labeling step, the gold nanoparticles were modified with a layer of 5,5′-dithiobis(succinimidyl-2-nitrobenzoate) (DSNB) to enable detection of the gold nanoparticles via Raman spectroscopy. A plot of the measured Raman intensity, corresponding to the vibrational mode for the DSNB symmetric nitro stretch, versus sample volume is shown. A “roll-off” in the Raman signal at larger sample volumes corresponds to approaching binding equilibrium between the h-IgG and membrane-bound antibody, a characteristic indicator of ND.

Note that elements in the figures are drawn for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

By way of context, the embodiments of the present invention are described within the framework of a sandwich immunoassay that uses antibody-modified gold nanoparticles (AuNPs) as labels for assay readout by surface-enhanced Raman spectroscopy (SERS). It should, however, be readily recognized that these embodiments apply beyond this illustrative example to include readout methods like fluorescence, surface plasmon resonance, chemiluminescence, electrochemistry, surface-enhanced infrared spectroscopy (SEIRA), ultraviolet-visible (UV-VIS) spectroscopy, and a number of other signal transduction methodologies.

This invention demonstrates a methodology that serves as a means to eliminate the need to meter an accurate and exceedingly small volume of the liquid sample through an SPME disk by configuring the microextraction to take advantage of the principles of negligible depletion (ND). The condition of ND, which will be more fully formulated shortly, occurs when the concentration of an analyte in a sample before passage through the SPME disk equals the concentration in the sample that exits the disk. In this scenario, the relevant analyte reaction (e.g., binding, complexation) reaches equilibrium so that the amount of extracted analyte remains proportional to the concentration in the sample but independent of the reaction kinetics and, consequently, sample volume. This approach can be more generally formulated to include other SPME embodiments such as, for example, systems that rely on the continuous passage of a sample through a small length of capillary tubing that is coated with a thin film of bonded phase. In most SPME applications, the condition of ND can be reached within a few minutes or less by rapidly passing relatively large volumes (several to tens of milliliters) of the sample through the membrane. However, the present invention specifically focuses on instances in which (1) the volume of sample available is much smaller than that typically required to reach the condition of ND; and (2) the rate of analyte uptake by the SPME membrane is much slower than the near-instantaneous processes more common to SPME. Stated differently, this means that the time to reach the condition of ND in small volume measurements may be several minutes, rather than several seconds, as the sample flow rate through the membrane must be markedly reduced for effective analyte extraction. Note that this invention can be coupled to methods wherein the bound analyte is measured directly on the SPME membrane and to methods wherein the bound analyte is measured after being eluted off the SPME membrane. Techniques for a direct measurement include, but are not limited to, fluorescence spectroscopy, surface-enhanced Raman spectroscopy (SERS), surface-enhanced infrared spectroscopy, ultraviolet-visible spectroscopy, diffuse reflectance spectroscopy, electrochemistry, quartz crystal microbalances (QCMs) and other acoustic wave devices, gas and liquid chromatography, mass spectrometry, NMR, and EPR techniques. Techniques for measuring the analyte concentration after elution off the SPME membrane include, but are not limited to, gas and liquid chromatography, mass spectrometry, NMR, and EPR techniques.

Immunoassays, which measures the presence or concentration of an analyte in a solution through the use of an antibody or an antigen, exemplify such a scenario due to the generally small sample volumes (≤0.500 mL), slow reaction kinetics, and challenging concentration ranges (fM-nM). To achieve ND for immunoassays and similar challenging analytical measurements, SPME membrane disks (and other embodiments) must be designed such that: (1) the effective volume of the solid capture surface or liquid bonded-phase is exceedingly small compared to the typical sample volume; (2) the sample residence time within the SPME disk or capillary is long in relation to the relevant analyte extraction reaction; and (3) sample contact with inactive areas of the SPME disk or membrane is minimized or prevented. In order to meet these requirements, the flow rate of the sample must be carefully controlled to increase the sample residence time, thereby increasing analyte extraction efficiency. In membrane-based SPME, this can be accomplished by, for example, careful selection of pore sizes, volume capacities, and composition of both the membrane disk and the underlying wicking pad. The extraction efficiency may also be increased by physically excluding sample flow through inactive areas of the membrane (i.e., areas not modified with analyte-specific antibody) forming confinement walls in the SPME material by using patterning methods such as inkjet printing, localized melting, or any other patterning method. This approach also increases resistance to sample flow, and consequently may also be used to control the sample flow rate independent of pore size. Note that SPME membranes can be fabricated from any number of materials typically used as reaction vessels for chemical and biochemical reactions and analyses, including but are not limited to: natural and human-made biomaterials, wood, paper, textiles (natural/synthetic), leather, glass, crystalline materials, biocomposite materials (bone/conch shell), plastics (natural/synthetic), rubber, (natural/synthetic), carbon, graphite, graphene, carbon nanotubes, and diamond materials, wax (natural/synthetic), metals, minerals, stone, concrete, plaster, ceramics, foams, salts, metal-organic frameworks (MOFs), covalent organic frameworks (COFs), nanomaterials, metamaterials, semiconductors, insulators, and composites of all of these.

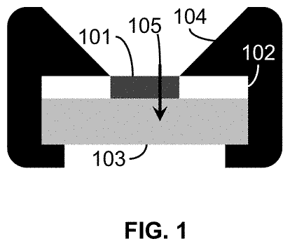

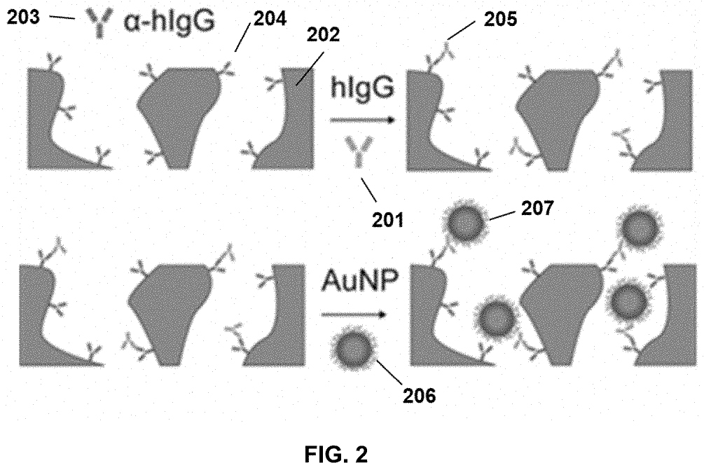

In addition to ensuring sufficiently low flow rates through the SPME membranes in order to reach to binding equilibrium, conditions that facilitate ND can be achieved by proper selection of the capture antibody and the surface density at which the antibody is immobilized on the membrane, forming the fundamental basis for the invention. The theoretical framework is developed below by first discussing the architecture of an SPME-based immunoassay and then considering chemical equilibrium theory for sandwich immunoassays to derive the conditions needed to reach ND. Note that the formulations that follow are for the assay of one analyte, but can be readily extended to yield a system of equations a multi-analyte design. For demonstrative purposes, FIG. 1 and FIG. 2 exemplify the assays of focus herein. FIG. 1 provides an illustrative example by way of a cross-section perspective of a typical, easily multiplexed design of a cartridge used in today's flow-through assays (FTAs). The cutaway view shows the four main components used in an FTA cartridge: a capture (reactive) address spotted on a disk (101), the membrane disk itself (102) a wicking pad (103), the membrane housing (104); the arrow depicts the direction of fluid flow (105).

FIG. 2 shows the steps and components for an approach to a sandwich immunoassay for a single analyte that uses surface-enhanced Raman scattering (SERS) as the optical readout method and human immunoglobulin G protein (h-IgG) as an example. Using this approach, the analyte (201) is extracted onto an SPME membrane (202) modified with an analyte-specific antibody (203) that forms the capture surface (204). The bound analyte (205) is labeled with antibody-modified nanoparticles (206)—usually consisting of gold, silver, or other plasmonically active material—to form the final sandwich complex (207) that can be detected spectroscopically.

For the example shown in FIG. 2 and, more generally, SPME-based immunoassays, the relationship between free and captured antigen is described by Eqn 1, where the analyte, which will be designated as an antigen (Ag) for context, is bound by the capture antibody (Ab_{cap}) to form a surface-bound antigen-antibody complex (AbAg). The equilibrium state of this reaction is expressed mathematically by Eqn 2; here, the association constant for antibody-antigen interaction, Kₐ₁, is expressed as the quotient of the product and reactant concentrations (denoted with brackets, [ ]) and can also be written as the ratio of the rates of the association and dissociation reactions. This equation assumes a 1:1 reaction stoichiometry for the antibody/antigen binding, but can readily be reformulated for other reaction stoichiometries. Eqn 2 can be converted to a form that reflects a heterogeneous assay, which upon rearrangement yields Eqn 3, which expresses the quantity of surface-bound antigen-antibody complex, Γ_{Ag} (mol/cm²), in terms of the following known parameters: the initial surface concentration of the capture antibody, Γ_{Cap,0} (mol/cm²); the initial concentration of antigen, C_{Ag} (mol/L); the volume of antigen solution, V_{Ag} (L); and the surface area of the SPME membrane modified with capture antibody (i.e., the capture “address”), A (cm²).

$$\begin{matrix}{Ab}_{Cap} + Ag ↔ AbAg & ( 1 ) \\ {K}_{a 1} = \frac{[ AbAg ]}{[ {Ab}_{Cap} ] [ Ag ]} & ( 2 ) \\ {K}_{a 1} = \frac{{Γ}_{Ag}}{( {C}_{Ag} - \frac{{Γ}_{AG} A}{{V}_{Ag}} ) ( {Γ}_{Cap , 0} - {Γ}_{Ag} )} & ( 3 )\end{matrix}$$

Finally, Eqn 3 can be rearranged to a quadratic expression (Eqn 4) that can be solved for the unknown variable Γ_{Ag}. The roots of Eqn 4, can be easily be solved numerically.

(AKₐ₁)Γ_{Ag}²+(−C_{Ag}Kₐ₁V_{Ag}−Γ_{Cap,0}AKₐ₁−V_{Ag})Γ_{Ag}+C_{Ag}Γ_{Cap,0}Kₐ₁V_{Ag}=0 (4)

For sandwich immunoassays, there is an additional equilibrium step between the surface-bound antigen and the secondary label used for quantitation by the strength of its signal upon readout. The equations describing the equilibrium between the surface-bound antigen and label of the sandwich immunoassay are derived in an analogous manner to those for the antigen-antibody steps. The quadratic form of the equation for the antigen-label reaction is shown in Eqn 5, where the unknown variable T_{Label} (mol/cm²), is dependent on the equilibrium surface concentration of bound antigen Γ_{Ag}; the label concentration, C_{L} (mol/cm³); and the equilibrium association constant for label binding, Kₐ₂ (cm³/mol).

(AKₐ₂)Γ_{Label}²+(−C_{L}Kₐ₂−Γ_{Ag}AKₐ₂−V_{L})+C_{L}Γ_{Ag}Kₐ₂V_{L}=0 (5)

From this system of equations, —it can be recognized that C_{Ag}, the unknown concentration of antigen in solution, can be determined exactly. The next steps recast the above treatment within the context of the conditions in which ND is operable when the sample volume is exceeding small and/or the rate of the extractive process is slow. The first step derives an equation for the surface concentration of analyte/antigen (Γ_{Ag}; mol/cm²) that binds to a membrane under the condition that the sample volume, V_{Ag} is very large; by extension, the total moles of antigen (n_{Ag}^{Total}) relative to the moles of antibody immobilized on the SPME membrane (Γ_{Cap,0}×A), is also very large. In this limiting case, the amount of antigen binding to the membrane-immobilized antibody (to form the antibody-antigen complex: n_{AgAb}^{Membrane}) has a negligible impact on the antigen solution concentration, as reflected in the mass balance equation given below in Eqn 6. Under these conditions, Eqn 3 can be reduced to a simpler form that similarly reflects insignificant antigen depletion (Eqn 7) by substitution of the

$$( {C}_{Ag}^{i} - \frac{{Γ}_{Ag} A}{{v}_{Ag}} )$$

term in the denominator with C_{Ag}ⁱ. In Eqn 7, Γ_{Ag}^{∞} denotes the surface concentration of bound antigen for a sample with infinite volume. Rearranging Eqn 7 to solve for Γ_{Ag}^{∞} yields Eqn 8.

$$\begin{matrix}{n}_{Ag}^{Solution} = {n}_{Ag}^{Total} - {n}_{AgAb}^{Membrane} \approx {n}_{Ag}^{Total} & ( 6 ) \\ {K}_{a 1} \approx \frac{{Γ}_{Ag}^{∞}}{{C}_{Ag}^{i} ( {Γ}_{Cap , 0} - {Γ}_{Ag}^{∞} )} & ( 7 ) \\ {Γ}_{Ag}^{∞} \approx \frac{{K}_{a 1} {C}_{Ag}^{i} {Γ}_{Cap , 0}}{1 + {K}_{a 1} {C}_{Ag}^{i}} & ( 8 )\end{matrix}$$

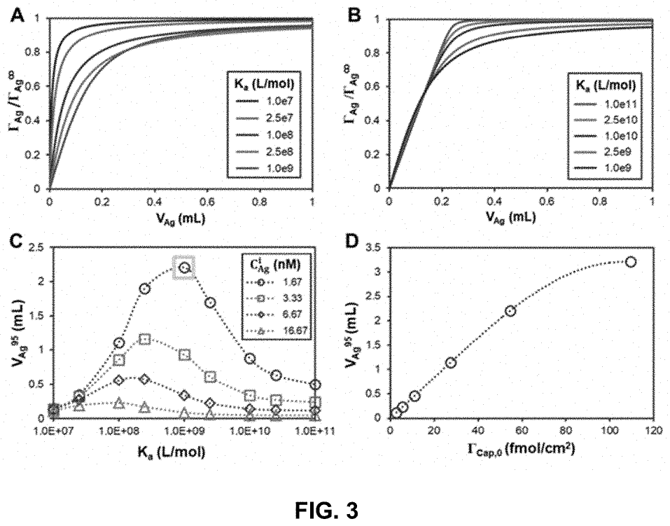

The next step is to define the conditions in which the antigen-binding step of the immunoassay approaches conditions of negligible depletion or, equivalently, Γ_{Ag}→Γ_{Ag}^{∞} with a commonly accepted and operative pre-defined tolerance value of 0.95. To do so, the ratio Γ_{Ag}/Γ_{Ag}^{∞} is calculated by dividing the appropriate root for Eqn 4 by Eqn 8. Of note, the root for Eqn 4 is complex, and so the explicit expression is necessarily omitted. Nevertheless, Γ_{Ag}/Γ_{Ag}^{∞} can easily be calculated by numerical methods. FIG. 3 displays the results for a set of calculations modeling the flow of an antigen solution through a 1.5×1.5 mm nitrocellulose membrane modified with a capture antibody. Inset A and inset B of FIG. 3 plot Γ_{Ag}/Γ_{Ag}^{∞} vs. sample volume (V_{Ag}) for a range of equilibrium association constants (Kₐ: 10⁷-10¹¹ L/mol), which reveal the significant impact that Kₐ has on the sample volume required to approach equilibrium (V_{Ag}⁹⁵; the volume at which Γ_{Ag}/Γ_{Ag}^{∞}=0.95). Note that the sample volumes required in most cases to reach Γ_{Ag}/Γ_{Ag}^{∞}=0.95 are within typical volumes used for diagnostic testing (≤0.500 mL). Inset C of FIG. 3, which plots V_{Ag}⁹⁵ for several different antigen concentrations (C_{Ag}ⁱ) and Kₐ values, sheds further light on the volumes required to approach the conditions of ND. For a given Kₐ, low antigen concentrations require greater sample volumes to approach ND. These simulations thus provide a framework for antibody selection based on Kₐ and anticipate analyte concentrations. It is noted that the required solution volumes are largest—in some cases, prohibitively so—for low and intermediate values of C_{Ag}ⁱ and Kₐ, respectively, which presents a challenging experimental obstacle. Referring to Eqn 8, Γ_{Ag}^{∞} is directly proportional to the starting antibody concentration, Γ_{Cap,0}. This proportionally implicitly links the extraction capacity of the SPME membrane to the total number of moles of Ag present in the sample, which is the product of the volume of the sample passed through the membrane and the concentration of the Ag in the sample.

Using the point in inset C of FIG. 3 surrounded by the gray box (C_{Ag}ⁱ=1.67 nM, Kₐ=1.0×10⁹) with an antibody surface concentration (Γ_{Cap,0}) of 54.7 fmol/cm², was examined for different values of Γ_{Cap,0}, with results plotted in inset D of FIG. 3. An imported conclusion from this theoretical treatment, highlighted in inset D of FIG. 3, is that Γ_{Cap,0} maybe intentionally reduced as part of the design of the SPME membrane, either by reducing the concentration of antibody in the precursor solution or through control of the volume deposited onto the SPME substrate, in order to lower the required sample volume. Although reducing Γ_{Cap,0} comes at the expense of signal in the final assay readout step, in many cases, ultrasensitive, portable analytical readout modalities (e.g., fluorescence and SERS spectroscopies) may compensate for the reduced signal. It should be noted that Γ_{Cap,0} may be selected based on the lowest analyte concentration that needs to be measured, as larger concentrations require lower sample volumes to reach equilibrium.

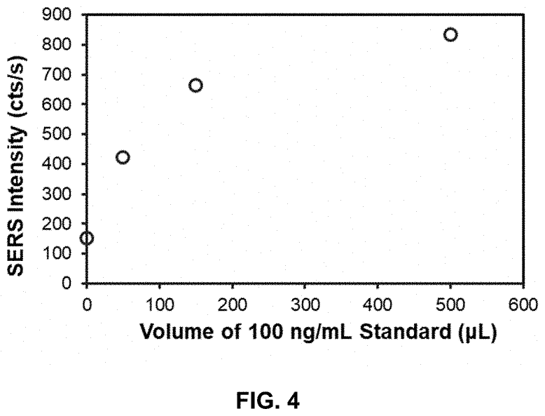

FIG. 4 provides supportive experimental confirmation of the above theoretical framework using an SPME membrane-based immunoassay for h-IgG with SERS readout. FIG. 4 plots the SERS intensity measured on a nitrocellulose SPME disk after exposure to different volumes of 100 ng/mL human IgG, with the SPME fabrication (i.e., Γ_{Cap,0}) and AuNP labeling steps carried out under the same conditions for all samples. The plot of SERS intensity vs. volume in FIG. 4 depicts a “roll-off” in the Raman signal, a profile characteristic of approaching the condition of negligible depletion. Because of the dependence of the uptake of antigen by the immobilized antibody, these types of profiles are observed for small sample volumes at low sample and label flow rates (e.g., 1-100 μL/min). Higher flow rates typically require much larger sample volumes in order to reach the ND condition. Note that these profiles can be reached by pretreating the SPME device with capture agent solutions having concentrations ranging from 0.05 to 5 mg/mL, depending on the magnitude of the binding affinity of the target with the immobilized antibody.

In the foregoing specifications, specific embodiments of the present invention have been described. However, various modifications and changes, such as the signal transduction method employed for assay readout, can be made without departing from the scope of the present invention as set forth in the claims below. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims, including any amendments made during the pendency of this application and all equivalents of those claims as issued.

What is claimed is:

1. A method of measuring the concentration of an analyte in a small volume of a liquid sample using an immunoassay based solid-phase microextraction (SPME) device, the method comprising the steps of:
flowing the liquid sample through the SPME device;
binding the analyte through antibodies/antigens immobilized on the SPME device;
obtaining a negligible depletion (ND) condition for the analyte within a predetermined time; and
measuring the concentration of the bound analyte using a readout technique.

2. The method of claim 1, wherein the volume of the liquid sample is less than 0.5 mL.

3. The method of claim 1, wherein the ND condition is obtained by controlling the flow rate of the liquid sample.

4. The method of claim 3, wherein the flow rate of the liquid sample is controlled by controlling the porosity and diameter of a flow channel of the SPME device.

5. The method of claim 4, wherein the diameter of the flow channels is controlled by forming confinement walls by inkjet printing, localized melting, or any other patterning method.

6. The method of claim 3, wherein the flow rate of the liquid sample is controlled by controlling the extractive capacity and composition of the SPME device.

7. The method of claim 3, wherein the flow rate of the liquid sample is controlled within a range from 1 to 100 μL/min.

8. The method of claim 1, wherein the ND condition is obtained by controlling the type and density of the antibodies/antigens immobilized on the SPME device.

9. The method of claim 8, wherein the density of the antibodies/antigens is controlled by pretreating the SPME device with capture agent solutions having concentrations ranging from 0.05 to 5 mg/mL.

10. The methods of claim 1, wherein the bound analyte is measured directly on the SPME device.

11. The methods of claim 1, wherein the bound analyte is measured after being eluted off the SPME device.

12. The methods of claim 1, wherein the readout technique includes but is not limited to fluorescence spectroscopy, surface-enhanced Raman spectroscopy (SERS), surface-enhanced infrared spectroscopy, ultraviolet-visible spectroscopy, diffuse reflectance spectroscopy, electrochemistry, quartz crystal microbalances (QCMs) and other acoustic wave devices, gas and liquid chromatography, mass spectrometry, NMR, and EPR techniques.

13. The methods of claim 1, wherein the SPME device is fabricated from materials typically used as reaction vessels for chemical and biochemical reactions and analyses, including but are not limited to: natural and human-made biomaterials, wood, paper, textiles (natural/synthetic), leather, glass, crystalline materials, biocomposite materials (bone/conch shell), plastics (natural/synthetic), rubber, (natural/synthetic), carbon, graphite, graphene, carbon nanotubes, and diamond materials, wax (natural/synthetic), metals, minerals, stone, concrete, plaster, ceramics, foams, salts, metal-organic frameworks (MOFs), covalent organic frameworks (COFs), nanomaterials, metamaterials, semiconductors, insulators, and composites of all of these.
